(19) United States
(12) Patent Application Publication
(10) Pub. No.: US 2005/0143771 A1
(43) Pub. Date: Jun. 30, 2005

(54) Lancing device with combination depth and activation control

(76) Inventors: Jeffrey T. Stout, Smyrna, GA (US); Ray Adams Lathrop, Atlanta, GA (US); Richard W. LeVaughn, Newnan, GA (US)

Correspondence Address: GARDNER GROFF, P.C., 2018 POWERS FERRY ROAD, SUITE 800, ATLANTA, GA, 30339, US

(21) Appl. No.: 11/001,771

(22) Filed: Dec. 2, 2004

Related U.S. Application Data
(60) Provisional application No. 60/526,213, filed on Dec. 2, 2003.

(51) Int. Cl. A61B 17/14; A61B 17/32

(52) U.S. Cl. 606/181

(57) ABSTRACT

A lancet, a housing for the lancent, and a combination depth and activation control assembly. The lancet includes a needle, a needle-holding section, and an operating section with an opening. A drive spring is mounted within the opening and between the lancet body and the housing. The control assembly includes a control member with a control interface knob and a control shaft. The control shaft has multiple stop surfaces that are alignable with an engagement surface on the lancet body to control the lancing depth. And the control shaft has a release surface that engages the release member to disengage the engagement member to activate the lancing stroke. Additional features includes a safety interlock for preventing accidental activiation of the lancing device, ridges on the housing for providing discrete locked and depth setting positions, and a keyed sterility cap for preventing accidental removal of the cap.

### CROSS-REFERENCE TO RELATED APPLICATION

This application claims the priority benefit of U.S. Provisional Patent Application Ser. No. 60/526,213, filed Dec. 2, 2003, the entirety of which is hereby incorporated herein by reference for all purposes.

### TECHNICAL FIELD

The present invention relates generally to medical devices and, more particularly, to a lancing device for penetrating the skin of a human or animal subject for sampling of blood and/or other body fluids.

### BACKGROUND OF THE INVENTION

Lancing devices are used to penetrate the skin of a subject and obtain a sample of blood or other body fluid, as in the testing of blood sugar levels by diabetics. Typically, a lancet having a sharp point is translationally mounted within a housing portion of a lancing device. The lancet is driven by a spring or other biasing means to cause the sharp point to extend a small distance through an opening in the housing and into the subject's skin, creating a wound from which the sample of body fluid is collected. The housing optionally includes a pressure surface for “pumping” the wound to enhance sample size, and may also incorporate a capillary tube or other sample collection media. The endcap of the housing or a portion of the housing adjacent the lancet opening may include an open window or a transparent section for viewing the sample collection site, and may also include one or more sample size indicators for comparing the size of a sample to a desired sample size. Example lancing devices are shown in U.S. Pat. No. 5,356,420; U.S. Pat. No. 5,397,334; and U.S. Pat. No. 5,439,473, all of which are hereby incorporated herein by reference.

Lancing devices typically are intended either for a single use or for multiple uses. Single-use lancing devices generally are disposed of after one use. For example, in a hospital or clinic, it is desirable to provide a single-use lancing device that can be used on a patient and then disposed of to eliminate any risk of infection to subsequent patients or caregivers from exposure to residual body fluids remaining on the lancing device. Accordingly, single-use lancing devices oftentimes include a disabling mechanism to prevent accidental or intentional re-use of the device. Various forms of disabling mechanisms are available, and are well known in the art. For example, the disabling mechanism may comprise a return spring for retracting the sharp point of the lancet back into the housing after a single use, break-away elements or a frangible link in the cocking or triggering mechanism to prevent re-arming or re-firing the device after a single use, a locking element, and/or a shield for blocking travel of the lancet.

Because single-use lancing devices normally are disposed of after one use, they generally are relatively simple in construction so that they can be economically manufactured in large quantities. To keep the design simple and economical, known single-use lancing devices do not include adjustability features. For example, known single-use lancing devices typically do not provide for adjustment of the depth of penetration of the lancet needle beneath the surface of the subject's skin. Accordingly, users of single-use lancing devices have little or no ability to adjust the depth and/or size of the wound in order to control the fluid sample size. While one user may be able to obtain a sufficient sample from a relatively small wound, another user who bleeds less freely may require a larger wound size to generate a sample of the same size. As a result, a user may be forced to suffer the pain of a lancet stick that is deeper than necessary to collect a sample of adequate size, or may need more than one lancet stick to generate a sufficient sample size. The lack of depth adjustment also renders previously known single-use lancing devices generally unsuitable for use in sampling from different body sites. For a given user, obtaining a sample of a specified size from a forearm sampling site typically requires a greater depth of penetration than obtaining the same size sample from a fingertip sampling site. Thus, previously known single-use sampling devices render it difficult or impossible for a user to obtain just the proper sample size from a single lancet stick, or to sample from different sites on the body.

Accordingly, a need exists for lancing devices providing depth adjustment of the lancet needle. In addition, it would be desirable for such lancing devices to be simple and economical in design, manufacture, and use so that they could be incorporated into single-use lancing devices. Furthermore, such lancing devices are needed that permit individual users to obtain the proper size and depth of wound for drawing fluid, without unnecessarily and repeatedly sticking oneself, without enduring the pain of deeper than needed sticks, and without the risk of contamination from any previous users. It is to the provision of lancing devices meeting these and other needs that the present invention is primarily directed.

### SUMMARY OF THE INVENTION

Briefly described, the present invention provides an improved lancing device that includes a housing, a lancet, and a combination depth and activation control assembly. The lancet includes a lancet needle, a lancet body that holds the needle, and a spring that propels the lancet body. The lancet is operable to travel from a charged position ready for activation, to an extended position with the lancet needle extending out of the housing, then to a retracted position with the lancet needle returned to within the housing.

In one aspect of the invention, the control assembly includes a control member, a release member, and an engagement surface on the lancet body. The control member includes a control interface and a control shaft. The control shaft has stop surfaces for depth control and an engagement surface for disengaging the release member from the engagement surface for activation control. In this configuration, the control member serves the dual purposes of setting the depth of the puncture and activating the lancet travel. Thus, the user manipulates a single component, the control member, to select the desired lancing depth and to initiate the lancing stroke.

In example embodiments, the control interface is a knob that rotates between a locked position and multiple depth setting positions. The control shaft extends from and rotates with the control knob, and is supported by a support arm that extends laterally from the housing. In addition, the control shaft has multiple stop surfaces, each offset a different thickness from the rotational axis of the shaft and positionable in alignment with the lancet body engagement surface. So by rotating the control knob, the user can selectively position the desired stop surface in alignment with the engagement surface to limit to travel of the lancet and thereby select the puncture depth of the lancet.

In addition, the release member is movable between a set position holding the lancet in the charged position and an activation position released from holding the lancet in the charged position. In the set position, the release member engages the lancet body engagement surface to hold the lancet in the charged position. Depressing the control knob moves the control shaft laterally so that it contacts and pushes the release member to the activation position. In this position, the release member is moved out of the way of the lancet body engagement surface, thereby releasing the lancet to be launched under the charge stored in the spring.

In another aspect of the invention, the lancet body includes an operating section with an inner wall forming an opening. The spring is positioned within the opening and mounted between the lancet body and the housing. For example, the spring may be mounted to the lancet body inner wall at a forward portion of the lancet body and to the support arm that extends laterally from the housing and into the opening. And the control shaft is supported by the arm so that the stop surfaces are alignable with the engagement surface at a rearward portion of the lancet body. In this way, the travel of the lancet is stopped by the impact of the lancet body engagement surface against one of the stop surfaces, and this impact is rearward and thus remote from the forward portion where the needle is, resulting in a less needle vibration and associated pain.

In yet another aspect of the invention, the lancing device includes a safety interlock feature. The safety interlock includes one or more lock tabs that align with and fit into one or more lock openings when the control knob is in the unlocked depth setting positions. But when the control knob is in the locked position, the lock tabs do not align with and do not fit into the lock openings, so the control knob cannot be depressed. In one example, one lock tab extends radially from the control shaft and the mating lock opening is formed in the housing and in communication with the control opening through which the control shaft extends. And in another example, two lock tabs extend from and are parallel to the control shaft axis, and two mating lock openings are formed in the housing in positions spaced apart from and on opposite sides of the control opening. In this way, the safety interlock prevents the control knob from being depressed when in the locked position, and permits the control knob to be depressed to activate the device when in one of the depth setting positions.

In still another aspect of the invention, the housing has a plurality of ridges or other protrusions with gaps between them. For example, four ridges may positioned in a generally circular arrangement so that when the control knob is in the locked position or one of the depth setting positions, the lock tabs are positioned in the gaps or the lock openings between the ridges. In addition, the housing may be provided with a deflection member that permits the control member to move laterally slightly out of the housing so it does not bind when engaging the ridges. In this way, the ridges, gaps, and deflection member provide a smoothly operable control member with discrete and readily perceivable depth setting and locked positions.

And in a further aspect of the invention, the sterility cap is keyed to the housing. For example, the sterility cap may have one or more tabs extending from it, and the housing may have one or more recessed portions that allow the tabs to fit through only when the tabs are aligned with the recessed portions, or vice versa. In this way, the sterility cap can only be removed after it has been manipulated by the user to align the key tabs and recessed portions. And the key tabs help reduce the spring load on the release member, support arm, and lancet body when the device is in the charged position during shipping and storage.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is an upper front perspective view of a lancing device according to a first example embodiment of the present invention, showing a housing, a lancet sterility cap, and the external components of a combination depth and activation control assembly.

FIG. 2 is a lower front perspective view of the lancing device of FIG. 1.

FIG. 3 is a rear perspective view of the lancing device of FIG. 1.

FIG. 4 is a rear exploded view of the lancing device of FIG. 1, showing a lancet and the internal components of the control assembly.

FIG. 5 is a front exploded view of the lancing device of FIG. 1.

FIG. 6 is a front view of the lancing device of FIG. 1, with a front housing piece and spring removed, showing the lancet in a charged position ready for activation.

FIG. 7 is a front view of the lancing device of FIG. 6, showing the lancet launched to an extended position for lancing the user's skin.

FIG. 8 is a front view of the lancing device of FIG. 6, showing the lancet returned by the spring to a retracted position within the housing.

FIG. 9 is a cross sectional view of the lancing device taken at line 9-9 of FIG. 1, showing the lancet in the charged position of FIG. 6, and the control assembly in a “deep” depth position and a “set” activation position.

FIG. 10 is a perspective cutaway view of the lancing device of FIG. 9, showing the control assembly in a “released” activation position and the lancet launched towards the extended position of FIG. 7.

FIG. 11 is a perspective view of the interior surface of the front housing piece of the lancing device of FIG. 1.

FIG. 11A is a side view of a detail of the front housing piece of the lancing device of FIG. 11.

FIG. 12 is a perspective view of the exterior surface of the front housing piece of FIG. 11.

FIG. 13 is a perspective view of a portion of the lancing device of FIG. 1, showing the control assembly in a “locked” activation position.

FIG. 14 is a front perspective view of a lancing device according to a second example embodiment of the present invention, showing a housing, a lancet sterility cap, and the external components of a combination depth and activation control assembly.

FIG. 15 is a rear perspective view of the lancing device of FIG. 14.

FIG. 16 is a front exploded view of the lancing device of FIG. 14, showing a lancet and the internal components of the control assembly.

FIG. 17 is a perspective view of a portion of the lancing device of FIG. 14, showing the control assembly in a “locked” activation position.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, the term “or” means “and/or,” and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

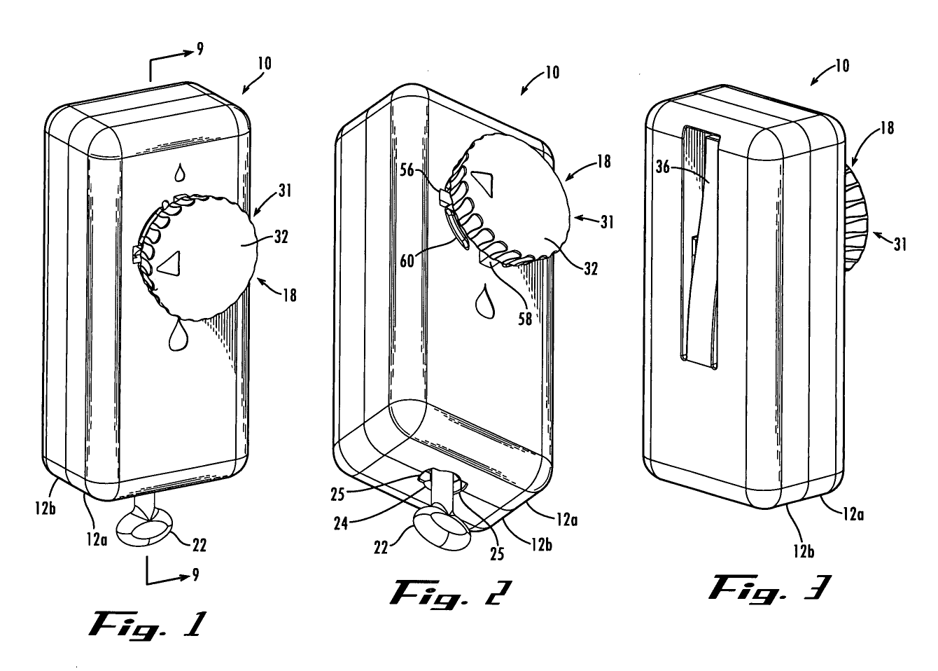

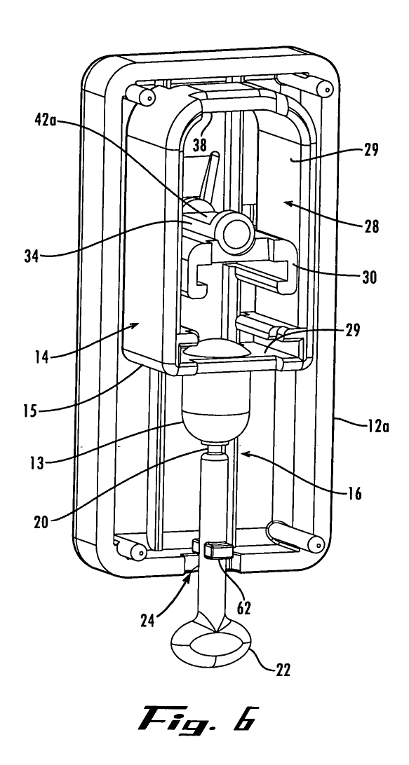

With reference now to the drawing figures, FIGS. 1-13 show a lancing device 10 according to a first example embodiment of the present invention. The lancing device 10 is a single use, disposable device. In alternative embodiments, the lancing device is adapted to accept replaceable lancets for use as a reusable lancing device.

As shown in FIGS. 1-5, the lancing device 10 includes a housing 12a and 12b (collectively referred to as “the housing 12”), a lancet 16, and a combination depth and activation control assembly 18 (the “control assembly” 18). The housing 12 may be made of two pieces 12a and 12b that couple together, as shown, or it may be made of more or fewer pieces, as desired. The lancet 16 includes a puncturing element such as a needle 20 for puncturing skin and a lancet body 14 that holds the needle 20. The lancet body 14 includes a needle-holding 13 section and an operating section 15. The needle 20 is covered by a removable sterility cap 22 that is easily removed to use the lancing device 10. The lancing device 10 is operable to extend the needle 20 through an opening 24 in the housing 12 to puncture a user's skin.

The housing 12, the lancet 16, and the combination depth and activation control assembly 18 may all be molded of a plastic material. If desired, the lancet body needle-holding section 13, the lancet body operating section 15, and the lancet needle 20 may be integrally molded into a single piece of plastic, or they may be separately made and assembled together, in which case the lancet needle may be made of metal or another material. It will be understood that other materials and fabrication techniques can be suitably employed, as would be recognized by a person of ordinary skill in the art.

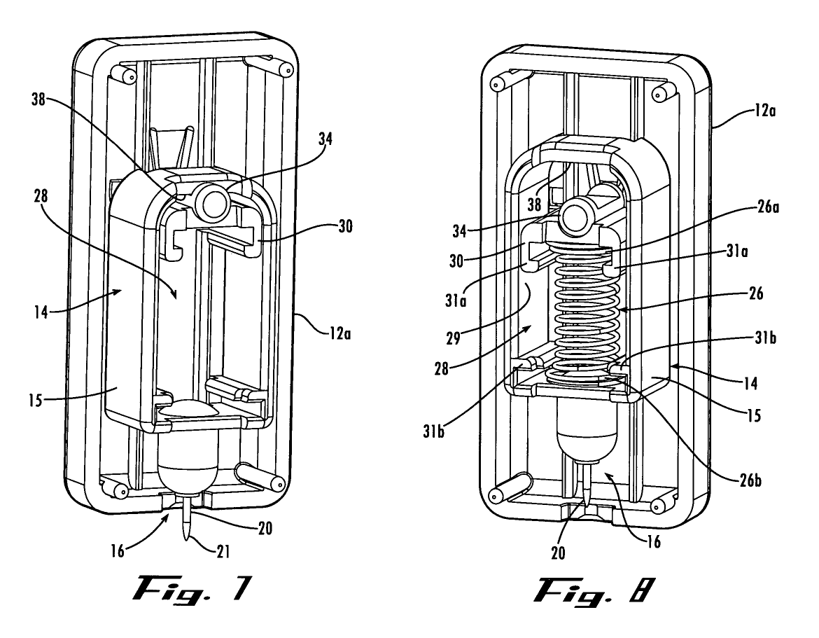

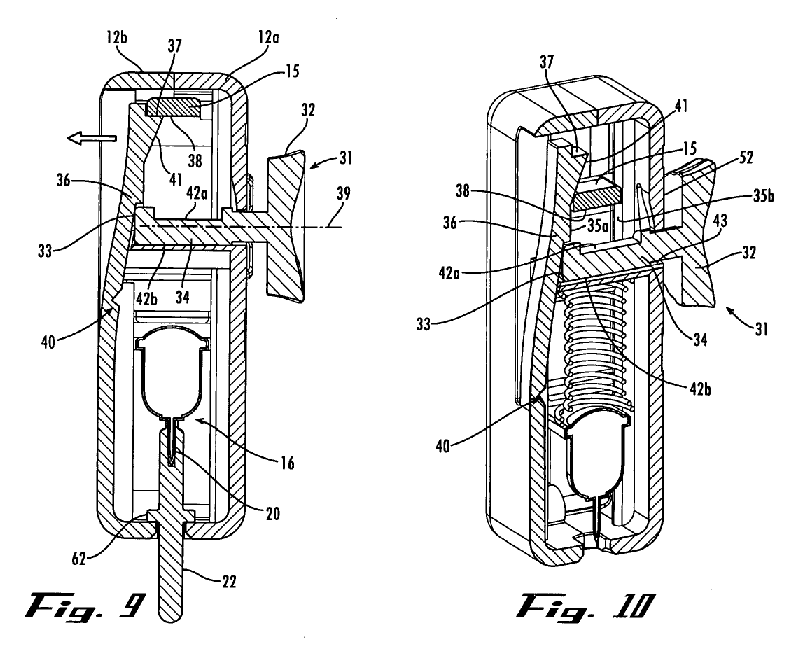

As shown in FIGS. 6-8, when the lancing device 10 is activated, the lancet 16 travels from a charged position (see FIG. 6) to an extended position (see FIG. 7) then to a retracted position (see FIG. 8). The lancet 16 is moved through these positions by the charge of an operating spring 26. Preferably, the spring 26 is positioned within an opening 28 defined by an inner wall 29 of the operating section 15 of the lancet body 14. And the spring 26 is preferably mounted between the lancet body inner wall 29 and an arm 30 extending laterally from the housing 12 and into the opening 28. For example, the spring 26 may be a coil spring that charges under compression and has flared ends or other portions, with a first flared portion 26a held by first flanges 31a on the arm 30 and a second flared portion 26b held by second flanges 31b on the lancet body inner wall 29. And the operating section 15 of the lancet body 14 may include a forward portion where the second flanges 31b and the needle-holding section 13 are positioned, and a rearward portion with an engagement surface 38 for limiting the lancet travel. (As used herein, “forward” means towards the lancing site and “rearward” means away from it.) In the depicted embodiment, the operating section 15 of the lancet body 14 is provided by a generally rectangular peripheral wall whose interior surface is the inner wall 29, which defines the opening 28 and the engagement surface 38.

In alternative embodiments, the lancet body may be provided by a solid body, two spaced apart panels with one central, four corner, or another arrangement of connecting posts, or other structures. The opening may be provided by a slot or other-shaped opening in the body. The spring may be provided by a torsion spring, leaf spring, other spring, other biasing element that functions like a spring, or combination thereof adapted for use to move the lancet. And the spring may be mounted between the lancet body and the housing by being integrally formed as a part of the body or housing, by flanges or couplings in other arrangements, or by other spring-mounting configurations known in the art.

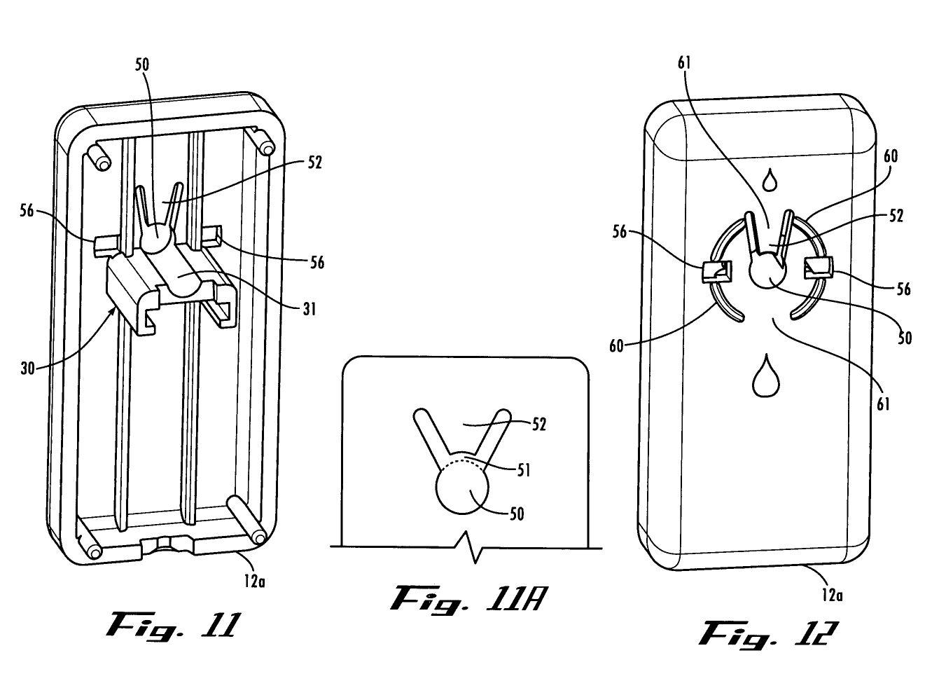

In the charged position of FIG. 6, the lancing device 10 is ready to be activated to launch the lancet 16. With the lancet body operating section 15 in the charged position as shown, the spring 26 (not shown) is compressed and thereby stores a charge. The control assembly 18 secures the lancet 16 in the charged position and is operable to release the lancet for launching by the spring 26, as described in detail with reference to FIGS. 9-13.

In the extended position of FIG. 7, the control assembly 18 has been activated to launch the lancet 16 to travel until a piercing end 21 of the needle 20 extends out of the housing 12 to pierce the skin. The travel of the lancet 16 is limited and stopped in the extended position when the engagement surface 38 on the inner wall 29 at the rearward portion of the lancet body operating section 15 contacts a stop surface 42. In the depicted embodiment, the stop surface 42 is defined on an adjustable control shaft to provide depth adjustment, though other stop surface arrangements may be used. In this configuration, the impact between mechanical members that stops the lancet travel occurs at the rearward portion of the lancet body 14, remote from the forward portion where the needle is. This provides greater stability and less vibration, and thus a cleaner and more painless puncturing, relative to conventional lancing devices in which the lancet travel is limited by the front exterior surface of the lancet body contacting the front interior surface of the housing.

As the lancet 16 travels from the charged position toward the extended position, the spring 26 is discharged to propel the lancet. Before the lancet 16 reaches the extended position, the spring 26 completely discharges its stored charge. Because the spring 26 is coupled to the lancet body 14, as the lancet body continues traveling to the extended position, the spring recharges under tension.

After the lancet 16 is momentarily stopped in the extended position, where the needle 20 is at its puncture depth, the lancet is now returned to the retracted position of FIG. 8 by the recharged spring 26. In the retracted position, the lancet needle 20 is safely within the housing 12 so that it will not stick a person handling the lancing device 10. In the retracted position, the spring 26 is in a neutral, uncharged state.

The lancing device 10 may be shipped and stored ready for use with the lancet 16 in the charged position of FIG. 6. Or the lancing device 10 may be shipped and stored with the lancet 16 in the retracted position of FIG. 8, in which case the user pushes in the sterility cap 22 to move the lancet to the charged position for use.

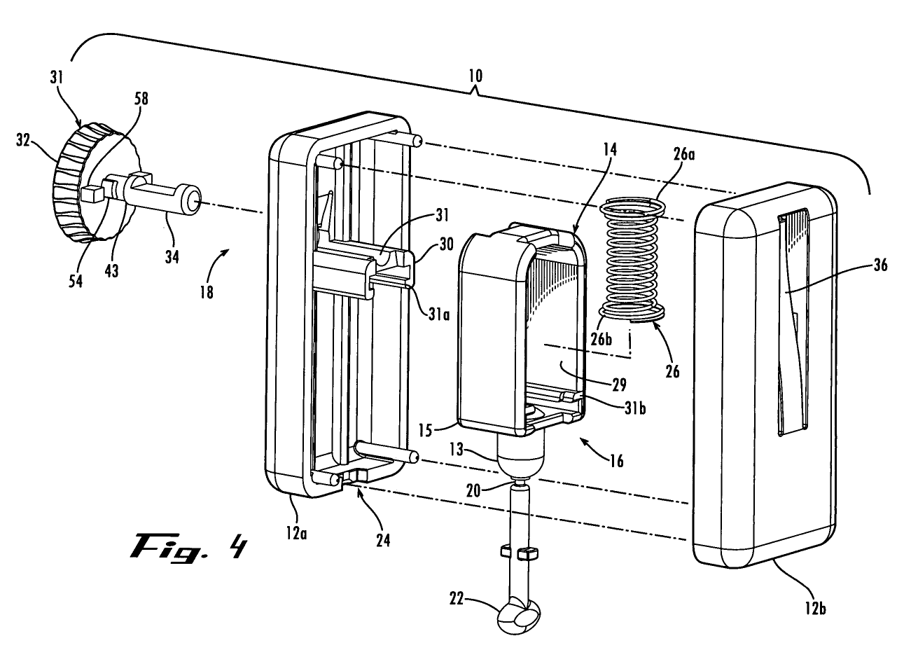

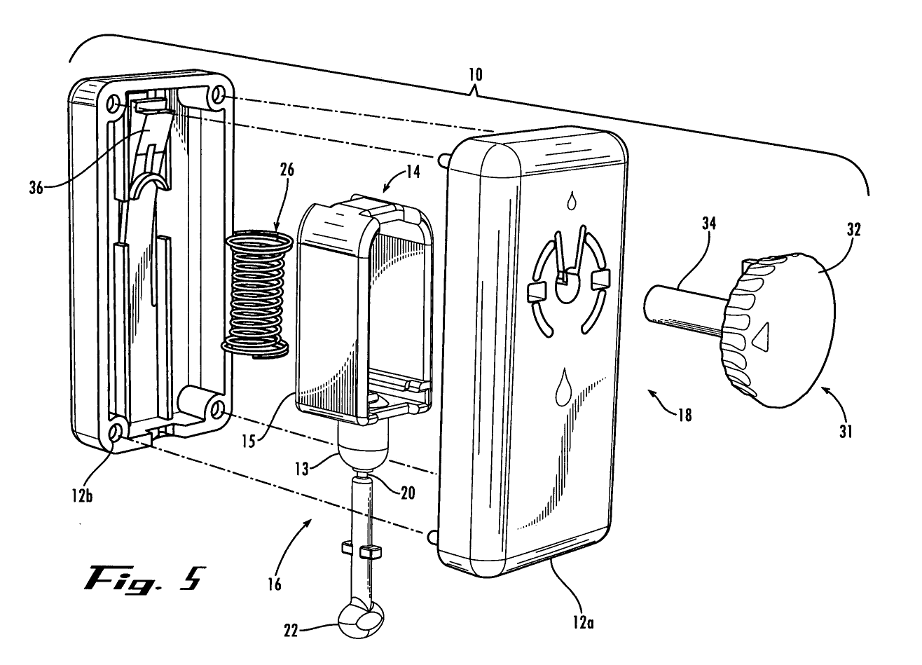

FIGS. 9-13 show details of the control assembly 18. First, details of the activation control will be described with reference to FIGS. 9 and 10. A control member 31 includes a control interface 32 (such the knob shown) and a shaft 34 extending from it and into the housing 12. The control shaft 34 is supported by the arm 30 and engages a release member 36. Preferably, the same arm 30 both supports the control shaft 34 and holds the spring 26, but separate structures may be provided. And the arm 30 may have a recess 31 (see FIG. 4) for cradling the shaft 34 and preventing or minimizing lateral movement. The release member 36 may be an integral part of the housing 12 or a separate piece that is assembled to the housing. In addition, the release member 36 has a catch surface 37 that engages the engagement surface 38 on the lancet body operating section 15, thereby holding the lancet 16 in the charged position. And the release member 36 is preferably biased toward the locked position and has a ramped section 41 adjacent the catch surface 37. When the lancet 16 is pushed inward to the charged position, the lancet body 14 engages the ramped section 41 and temporarily moves the release member out of the way.

The release member 36 is movable between a set position holding the lancet 16 in the charged position and an activation position released from holding the lancet 16 in the charged position. The release member 36 is preferably a leaf spring, pivotal lever, plunger, or other structure that is movable between the set position and the activation position in response to movement of the control shaft 34 between a set position and an activation position. In addition, the release member 36 may have a generally vertical guide surface 35a, and the housing 12 may have a generally vertical surface section 35b that is positioned on the opposite side of the lancet body 14 to help guide the lancet body, and thus the lancet needle 20, thereby reducing vibration and associated pain.

In the depicted embodiment of FIG. 9, for example, the release member 36 and the control shaft 34 are in the set position. Depressing the control knob 32 moves the control shaft 34 laterally into the housing 12. The control shaft 34 has a release surface 33 on the opposite side of the lancet body 14 from the control knob 32. Moving the control shaft 34 causes the release surface 33 to push the release member 36 (in the direction indicated by the directional arrow) to the activation position (not shown). In this position, the catch surface 37 of the release member 36 is moved out of the way of the engagement surface 38 on the lancet body operating section 15, thereby releasing the lancet 16 to be launched under the charge stored in the spring 26. When the control knob 32 is released by the user, the control shaft 34 and release member 36 return to their original positions, as shown in FIG. 10.

In addition, the release member 36 may have a weak segment 40 such as a notch, recess, or other thinner wall portion. The weak segment 40 is designed to fail upon activation so that the lancing device 10 can not be charged and used again. It will be understood by persons skilled in the art that other disabling mechanisms can be used.

Referring additionally to FIGS. 11-13, there are shown additional details of the activation control and details of the depth control of the control assembly 18. The depth control is provided by two stop surfaces 42a and 42b (collectively, the “stop surfaces 42”). Preferably, the stop surfaces 42 are defined on or in the control shaft 34 and have different thicknesses as measured from the rotational axis 39 of the control shaft. The stop surfaces 42 may be provided by recesses in or protrusions on the control shaft 34. In alternative embodiments, the control shaft is provided with three or another number of stop surfaces on or in the control shaft, thereby providing more lancet puncture depth adjustment positions. And other alternative embodiments include a single depth lancing device with the single stop surface defined by the control knob, the arm, or another structural component.

In the depicted embodiment, the control shaft 34 has a first stop surface 42a defined by a recessed surface and a second stop surface 42b defined by the outer surface of the shaft, thereby providing two depth settings. The second stop surface 42b is thicker (farther from the rotational axis 39 of the control shaft 34) than the first stop surface 42a. Thus, when the control knob 32 is turned so that its control indicator 44 aligns with a deep depth indicator 46 on the housing 12, the control shaft 34 is positioned with the first stop surface 42a facing and aligned with the engagement surface 38 of the lancet body 14 (see FIG. 6). In this position, the lancet 16 is free to travel farther (see FIG. 7) than it would in the shallow depth setting.

In the shallow depth setting (not shown), the control knob 32 is turned so that the control indicator 44 aligns with a shallow depth indicator 48 on the housing 12. In this setting, the control shaft 34 is positioned with the thicker second stop surface 42b facing and aligned with the engagement surface 38 of the lancet body 14. In this position, the lancet 16 travel is stopped sooner by the thicker stop surface 42b, resulting in a shallower puncture depth.

In this configuration, the control assembly 18 includes common structures for controlling both the puncture depth of the lancet needle 20 and the activation of the lancing stroke. In particular, the control assembly 18 includes the control member 31 which has multiple stop surfaces 42 for stopping the lancet travel at different points (to control the puncture depth) and a release surface 43 for engaging the release member 36 to disengage it from the lancet body 14 (to control the lancet activation). By providing common structures that function for dual purposes, the number of parts is reduced and the ease of manufacture is increased. This results in a simpler, less costly device.

The activation control further includes a unique safety interlock feature wherein the control knob 32 has a locked position and one or more depth setting positions (e.g., the shallow and deep settings described herein). With the control knob 32 in the locked position, it cannot be depressed to activate the lancet 16. And with the control knob 32 moved to one of the depth setting positions, it can be depressed to activate the lancet 16. In this way, the lancing device 10 remains locked and cannot be accidentally activated until the user selects a depth setting. Conventional single use lancing devices do not have a safety interlock feature and are shipped ready for firing, so occasionally a lancet is inadvertently activated before intended.

In the lancing device 10 shown, for example, the housing 12 has a control opening 50 through which the control shaft 34 extends, with the control opening and control shaft preferably generally circular in cross-section. The housing 12 has at least one lock opening 51 in communication with the control opening (see FIG. 11A). The lock opening 51 is adjacent a deflection member 52, which has a radiused edge that is coaxial with the control opening 50. And the control shaft has at least one lock tab 54 (see FIG. 4) extending radially from it. When the control knob 32 is in the locked position (see FIG. 13), the lock tab 54 does not align with the lock opening 51, so the control knob cannot be depressed to activate the lancet 16. But when the control knob 32 is turned (e.g., 90 degrees) to one of the depth setting positions, the lock tab 54 aligns with and can fit through the lock opening 51, so the control knob can be depressed to activate the lancet 16. It will be understood that another number of lock tabs and openings can be provided.

As another example, in the lancing device 10 shown, the housing 12 additionally has two lock openings 56 spaced apart from and on opposite sides of the control opening 50. And the control knob 32 has two lock tabs 58 extending from it generally parallel to the control shaft axis 39. When the control knob 32 is in the locked position (see FIG. 13), the lock tabs 58 do not align with the lock openings 56, so the control knob cannot be depressed to activate the lancet 16. But when the control knob 32 is turned to one of the depth setting positions, the lock tabs 58 align with and can fit through the lock openings 56, so the control knob can be depressed to activate the lancet 16.

In addition, the housing 12 may have a plurality of protrusions 60 extending from its outer surface with gaps 61 between them. For example, the protrusions 60 may be provided by four ridges positioned in a generally circular arrangement with the two gaps 61 and the two lock openings 56 interposed between the ridges. In this way, when the control knob 32 is in the locked position or one of the depth setting positions, the lock tabs 58 are positioned in the gaps 61 or in the lock openings 56 between the ridges 60. This creates discrete positions that are readily sensible (e.g., snap sound and/or tactile perception) by the user.

In order to maintain a nice tight fit, but not a binding fit, between the ridges 60 and the lock tabs 58 when turning the control knob 32, at least one deflection member 52 may be provided. The deflection member 52 is defined by two slits or other spaced-apart openings in communication with the control opening 50. The deflection member 52 functions as a leaf spring that deflects slightly outward when the lock tabs 58 engage the ridges 60, thereby permitting the control shaft 34 to back slightly out of the housing 12. The control shaft 34 has a collar 43 that holds it in place so that it can rotate but not move laterally out of the housing 12, except by movement of the deflection member 52 (see also FIGS. 4 and 10). In alternative embodiments, a spring or other biasing structure may be provided for this function.

In addition, the sterility cap 22 is preferably keyed to the housing 12 so that the cap can only be removed after it has been manipulated by the user. For example, the sterility cap 22 may have sheath portion with that extends into the housing 12 and one or more tabs 62 or other male key surfaces extending from it (see, e.g., FIGS. 6 and 9). And the housing opening 24 may be sized and shaped to receive the cap 22 through it and have one or more recessed portions 25 or other female key surfaces that allow the tabs 62 to fit through only when the tabs are aligned with the recessed portions (see, e.g., FIG. 2). Or vice versa—the housing 12 may have the tabs and the sterility cap 22 may have recessed portions that are alignable with the tabs. The lancing devices are provided with the tabs 62 not in alignment with the recessed portions 25 so that, in order to remove the sterility cap 22 to use the lancing device 10, the user must rotate the cap until the tabs 62 and the recessed portions 25 are aligned. This prevents accidentally removing the sterility cap 22 before intended. And with the key tabs 62 not aligned with the key recessed portions 25, the key tabs contact the interior forward wall of the housing 12. This prevents the lancet 16 from being launched prematurely and also helps reduce the spring load on the release member 36, support arm 30, and lancet body 14 when the lancing device 10 is in the charged position during shipping and storage.

It will be understood that several of the unique features of the lancing device 10 may be provided independently of each other. For example, the present invention includes lancing devices that have the control assembly but not the safety interlock or the keyed sterility cap, as well as lancing devices that have the safety interlock and/or the keyed sterility cap but not the control assembly.

To use the lancing device 10, a user rotates the control knob 32 from the locked position to the desired puncture depth setting position. Then the user turns the sterility cap 22 until the keying permits its removal, removes the cap from the lancet needle 20, and discards the cap. Next the user positions the lancing device 10 against the skin at the desired lancing site and depresses the control knob 32 to activate the device. Thus, the user manipulates a single component, the control member 31, to select the desired lancing depth (by rotation) and to initiate the lancing stroke (by lateral movement/pushing inward). After use, the user disposes of the used lancing device 10.

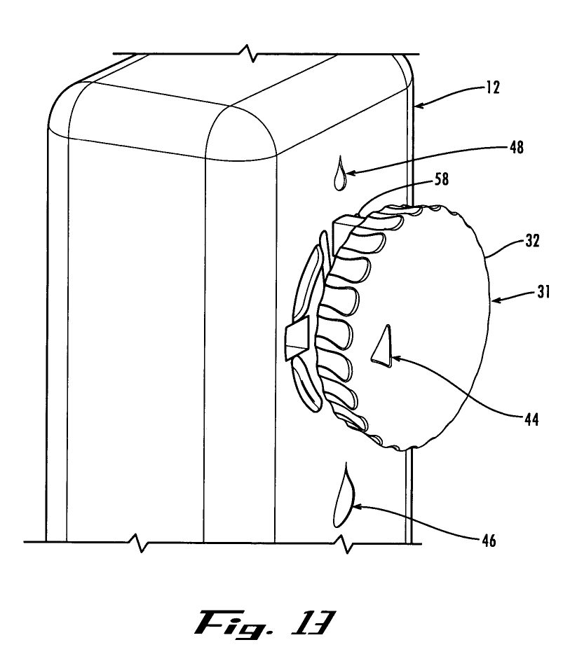

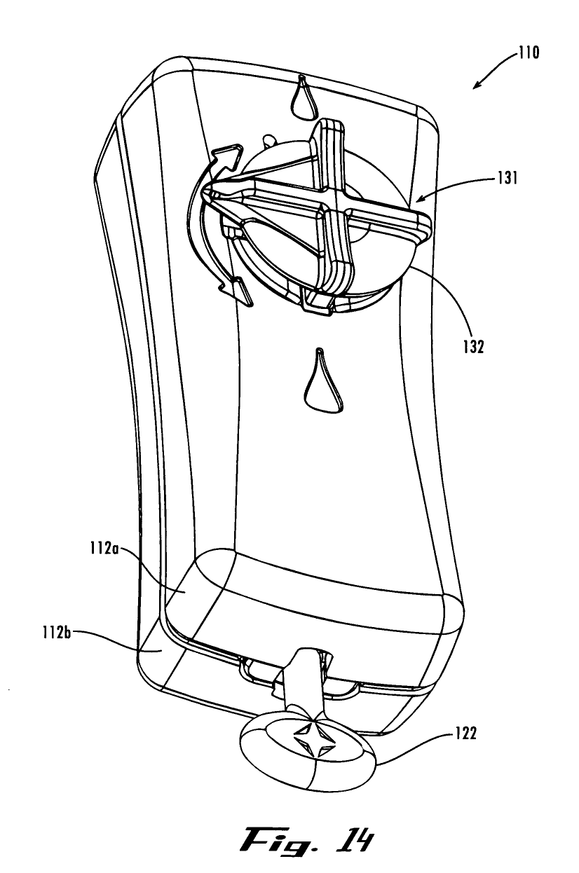

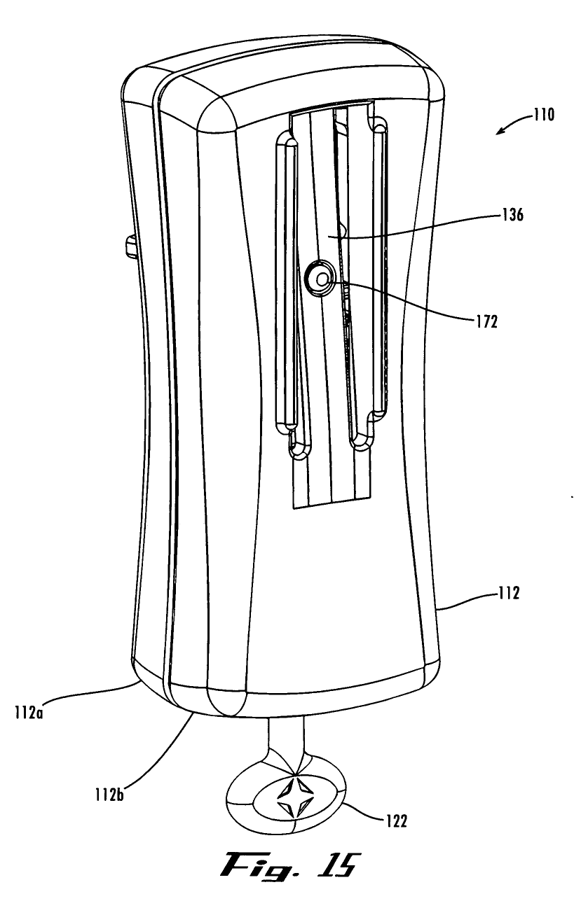

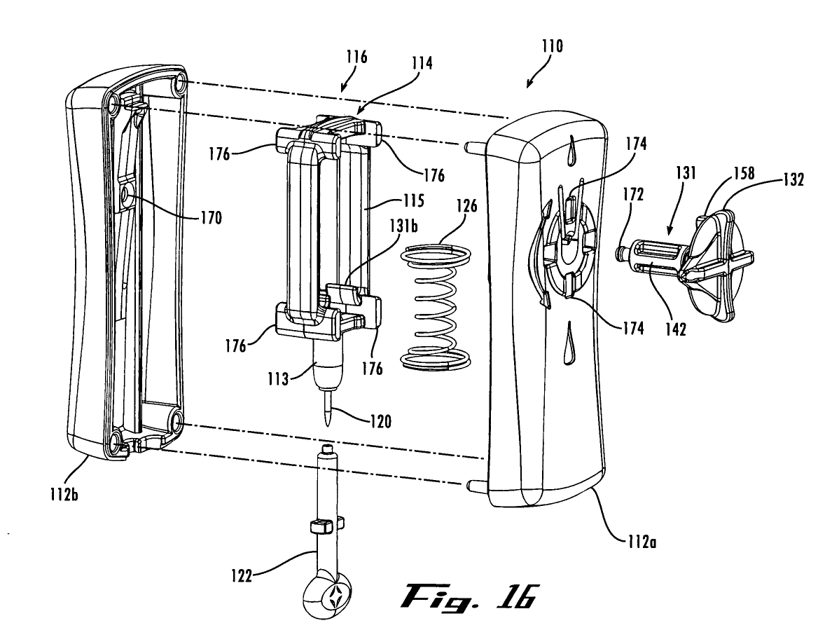

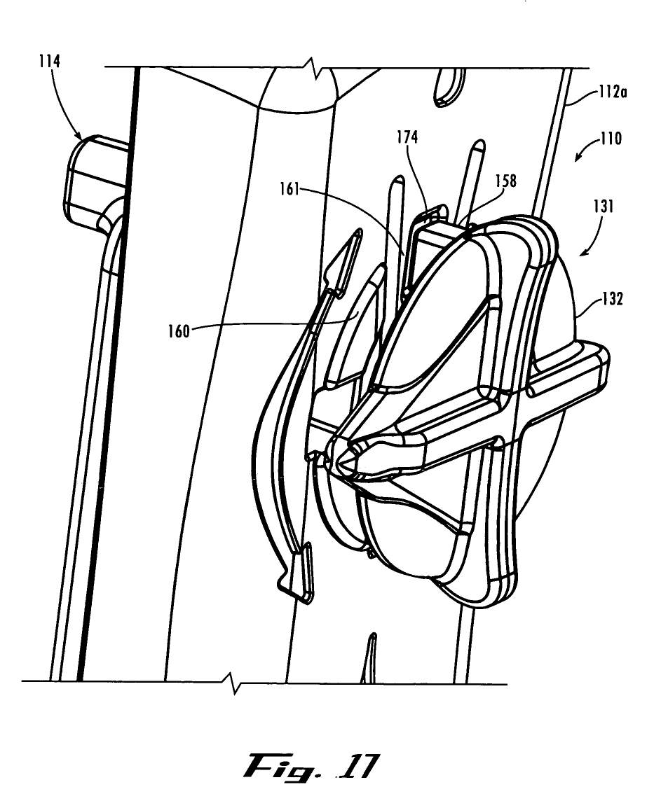

Turning now to FIGS. 14-17, there is shown a lancing device 110 according to a second example embodiment of the present invention. Similarly to the lancing device 10 of the first example embodiment, the lancing device 110 includes a housing 112a and 112b (the “housing 112”), a lancet 116, and a combination depth and activation control assembly 118 (the “control assembly” 118). The lancet 116 includes a needle 120 with a sterility cap 122 and a lancet body 114 with a needle-holding 113 section and with an operating section 115 holding the spring 126. And the control assembly 118 includes the control member 131, the release member 136, and the engagement surface 138 of the lancet body 114. The control member 131 includes the control knob 132 and the control shaft 134 with the stop surfaces 142 and release surface 143.

In this embodiment, however, the control shaft 134 is coupled to the release member 136 so that they move together. For example, the control shaft 134 may extend through an aperture 170 in the release member 136 and have a retainer head 172 to secure it in place with the engagement surface 138 contacting the release member. In alternative embodiments, the control shaft 134 is coupled to the release member 136 by a bendable or expandable member at the end of the control member, by a threaded nut that screws onto mating threads on the control shaft, or by other couplings known in the art.

In addition, the housing 112 has recesses 174 in the gaps 161 between the ridges 160, with the recesses sized and shaped to receive the lock tabs 158. This feature further enhances the user's tactile perception of the discrete locked and depth positions.

Furthermore, the housing 112 has a slightly bowed-in shape for enhanced ergonomics. And the lancet body 114 has a slightly different design, including angled flanges 131b for retaining the spring 126 and the addition of guide wings 176 for enhanced guidance of the lancet 116 within the housing 112.

Accordingly, the present invention provides a number of advantages not found in known prior art devices. The combination depth and activation control assembly is operable to control both the puncture depth and the activation of the device, thereby enabling a low-cost disposable lancing device with depth adjustment capability. The safety interlock feature prevents accidental activation of the lancing device before intended. And the keyed sterility cap requires manipulation of the cap to remove it, thereby preventing accidental removal of the cap before intended.

While the invention has been described with reference to preferred and example embodiments, it will be understood by those skilled in the art that a variety of modifications, additions and deletions are within the scope of the invention, as defined by the following claims.

1. A lancing device, comprising:
a lancet movable from a charged position ready for use to an extended position for puncturing skin;
a release member that engages and retains the lancet in the charged position; and
a combination depth and activation control assembly including a control member having a plurality of stop surfaces and a release surface, wherein the control member is movable to position the stop surfaces to stop the lancet at a plurality of different depths, and the control member is movable to position the release surface in engagement with the release member to release the lancet to travel to the extended position.

2. The lancing device of claim 1, wherein the control member includes a control shaft that defines the stop surfaces, and the control shaft rotates to position one or another of the stop surfaces to contact and stop the lancet.

3. The lancing device of claim 2, wherein a first one of the stop surfaces is spaced closer to a rotational axis of the control shaft than a second one of the stop surfaces, wherein the first stop surface stops the lancet at a longer travel and deeper puncture depth than the second stop surface.

4. The lancing device of claim 1, wherein the control member includes a control shaft that defines the release surface, and the control shaft moves axially to push the release member out of engagement with the lancet to activate the lancing device.

5. The lancing device of claim 2, wherein the control member includes a control shaft that defines the release surface, and the control shaft moves axially to push the release member out of engagement with the lancet to activate the lancing device.

6. The lancing device of claim 1, wherein the lancet has an engagement surface, the release member engages the engagement surface to retain the lancet in the charged position, and the stop surfaces engage the engagement surface to stop the lancet in the extended position.

7. The lancing device of claim 1, further comprising a spring that propels the lancet from the charged position to the extended position, wherein the lancet includes a lancet body that defines an opening, and the spring is positioned within the opening.

8. The lancing device of claim 7, further comprising a support arm extending into the opening, wherein the spring is mounted to the lancet body and the support arm.

9. The lancing device of claim 8, wherein the support arm supports the control shaft.

10. The lancing device of claim 8, wherein the opening is defined by an inner wall of the lancet body, the inner wall defines an engagement surface at a rearward portion of the lancet body, and the stop surfaces engage the engagement surface to stop the lancet in the extended position.

11. The lancing device of claim 1, further comprising a housing for the lancet, wherein the housing defines at least one lock opening, the control member includes a control interface member with at least one lock tab extending therefrom, and the control interface is movable from a locked position with the lock tab not aligned with the control opening to a plurality of depth setting positions with the lock tab aligned with and receivable in the control opening to permit movement of the control member.

12. A lancing device, comprising:
a lancet movable from a charged position ready for use to an extended position for puncturing skin, the lancet having a lancet body defining an engagement surface;
a release member movable from a set position engaging the lancet body engagement surface and retaining the lancet in the charged position to an activation position releasing the lancet to travel to the extended position; and
a combination depth and activation control assembly including a control member having a control interface and a control shaft extending therefrom, the control shaft having a release surface and first and second stop surfaces, wherein the control interface is rotatable to rotate the control shaft to position either the first stop surface or the second stop surface to contact the lancet body engagement surface to stop the lancet in the extended position at a first or a second puncture depth, and wherein the control interface is depressible to axially move the control shaft to push the release surface against the release member to move the release member from the set position to the activation position to activate the lancing device.

13. The lancing device of claim 12, wherein the first stop surface is spaced closer to a rotational axis of the control shaft than the second stop surface, wherein the first stop surface stops the lancet at a longer travel and deeper puncture depth than the second stop surface.

14. The lancing device of claim 12, wherein the first and second stop surfaces are positioned between the release surface and the control interface.

15. The lancing device of claim 12, further comprising a spring that propels the lancet from the charged position to the extended position, wherein the lancet body has an inner wall that defines an opening and that defines the engagement surface at a rearward portion of the lancet body, and the spring is positioned within the opening.

16. The lancing device of claim 15, further comprising a support arm extending into the opening, wherein the spring is mounted to the lancet body and the support arm, and the support arm supports the control shaft.

17. The lancing device of claim 12, wherein the lancet body includes a peripheral wall that has a generally rectangular shape and that defines the opening.

18. The lancing device of claim 12, wherein the release member is biased toward the set position, defines a catch surface that engages the lancet body engagement surface in the charged position, and defines a ramped section that engages the control shaft release surface to deflect the release member from the set position when moving the lancet to the charged position.

19. The lancing device of claim 12, wherein the release member includes a weak segment that fails upon movement to the activation position to disable the lancing device.

20. The lancing device of claim 12, further comprising a housing for the lancet, wherein the housing defines at least one lock opening, the control interface has at least one lock tab extending therefrom, and the control interface is movable from a locked position with the lock tab not aligned with the control opening to first and second depth setting positions with the lock tab aligned with and receivable in the control opening to permit the axial movement of the control member.

21. A lancing device, comprising:
a lancet movable from a charged position ready for use to an extended position for puncturing skin, the lancet including a body and a puncturing element extending from a forward portion of the lancet body, the lancet body having an inner wall that defines an opening and defines an engagement surface at a rearward portion of the lancet body;
a spring that propels the lancet forward from the charged position to the extended position, wherein the spring is positioned within the opening; and
a control member defining at least one stop surface that engages the lancet body engagement surface to stop the lancet in the extended position, wherein the impact of the lancet member engagement surface and the stop surface is remote from the puncturing element.

22. The lancing device of claim 21, further comprising a support arm extending into the opening, wherein the spring is mounted to the lancet body and the support arm and the support arm supports the control member.

23. The lancing device of claim 21, further comprising a release member that engages the lancet body engagement surface and retains the lancet in the charged position.

24. A lancing device, comprising:
a housing defining at least one lock opening;
a lancet movable from a charged position ready for use to an extended position for puncturing skin;
a control member having a control interface member with at least one lock tab extending therefrom, wherein the control interface is movable from a locked position with the lock tab not aligned with the lock opening to at least one unlocked position with the lock tab aligned with and receivable in the lock opening to permit the control member to move to activate the lancing device.

25. The lancing device of claim 24, wherein the housing defines a plurality of ridges with gaps therebetween, the gaps positioned to receive the lock tab when the control interface is in the locked position and the at least one unlocked position to provide discrete positioning of the control interface.

26. The lancing device of claim 25, wherein the housing defines a deflection member that engages and is deflected by the lock tab when the control interface is moved from the locked position to the at least one unlocked position.

27. A lancing device, comprising:
a housing defining at least one lancing opening;
a lancet having a body and a puncturing element extending from the body, wherein the lancet is movable from a charged position within the housing to an extended position with the puncturing element extending though the lancing opening for puncturing skin; and
a sterility cap positioned on the puncturing element and removable for use, wherein the sterility cap has a sheath portion that extends into the housing through the lancing opening, the sterility cap defines at least one first key element on the sheath portion within the housing and the housing defines at least one second key element at the lancing opening, and the sterility cap is rotatable from a locked position with the first and second key elements not in alignment to an unlocked position with the first and second key elements in alignment and permitting the sterility cap to be removed from the puncturing element.
